(19) United States
(12) Patent Application Publication
(10) Pub. No.: US 2025/0065115 A1
(43) Pub. Date: Feb. 27, 2025

(54) THERAPEUTIC SYSTEMS AND METHODS USING ELECTROPORATION

(71) Applicant: Mayo Foundation for Medical Education and Research, Rochester, MN (US)

(72) Inventors: Barham K. Abu Dayyeh, Rochester, MN (US); Jason A. Tri, Rochester, MN (US); Samuel J. Asirvatham, Rochester, MN (US); John J. Mullon, Rochester, MN (US); Ryan M. Kern, Rochester, MN (US)

(21) Appl. No.: 18/719,045

(22) PCT Filed: Jan. 11, 2023
(86) PCT No.: PCT/US2023/010564

§ 371 Date: Jun. 12, 2024

Related U.S. Application Data
(60) Provisional application No. 63/298,815, filed on Jan. 12, 2022.

(51) Int. Cl. A61N 1/32

(52) U.S. Cl. CPC A61N 1/327

(57) ABSTRACT

Devices, systems, and methods described in this disclosure can be used to deliver electroporation and adjunct therapies to treat anastomotic leaks and the resulting abscess cavities around the GI tract, within the thoracic cavity, and elsewhere within the human body. The thermal or non-thermal electroporation therapy can be delivered using the devices, systems, and methods described herein. In some embodiments, the electroporation energy is delivered to achieve sterilization and promote healing of spaces such as abscess cavities.

### CROSS-REFERENCE TO RELATED APPLICATIONS

This application claims the benefit of U.S. Provisional Application Ser. No. 63/298,815, filed Jan. 12, 2022. The disclosure of the prior application is considered part of (and is incorporated by reference in) the disclosure of this application.

### BACKGROUND

### 1. Technical Field

This document relates to devices and systems for delivering electroporation therapy or pulsed electric field and methods for their use. For example, this document relates to devices, systems, and methods for delivering electroporation and adjunct therapies to treat anastomotic leaks and the resulting abscess cavities around the gastrointestinal tract, thoracic cavity, and elsewhere within the human body. In addition, it can be used to approximate a hollow structure within the thoracic or abdominal cavities, such as pleural or peritoneal space to apply a pulsed electric field that can decrease or eliminate the infectious load within the structure and or enhance the adhesion and collapse of the structure.

### 2. Background Information

Anastomotic leaks are defined as a leak of luminal contents from a surgical anastomosis following gastrointestinal (“GI”) or thoracic surgery. The leaked content often causes systemic infection, sepsis, and results in the formation of abscess cavities. Incidence rates of anastomotic leaks or iatrogenic leaks ranges from 2-10% depending on the type of surgery; thus, presenting significant burden to the affected patient and healthcare system.

Current management strategies call for either morbid surgical management, or minimally invasive, but inefficient, endoscopic or percutaneous drainage with passive catheters. However, these therapies are inefficient, because the sponge requires multiple changes as it gets soiled and ineffectual from luminal GI secretion and contents entering the leak cavity given no barrier effect in these systems. In addition, none of the existing technologies is capable of actively managing or decreasing the infectious load within the cavity beyond application of suction.

### SUMMARY

This document describes devices and systems for delivering electroporation therapy or pulsed electric field and methods for their use. For example, this document describes devices, systems, and methods for delivering electroporation and adjunct therapies to treat anastomotic leaks and the resulting abscess cavities around the GI tract, within the thoracic cavity, and elsewhere within or on the human body. The thermal or non-thermal electroporation therapy can be delivered using the devices, systems, and methods described herein. In some embodiments, the electroporation energy is delivered to achieve sterilization and healing of spaces such as abscess cavities, peritoneal, pleural, or peri-cardiac space.

In one aspect, this disclosure is directed to a medical device system that includes an introducer sheath configured for percutaneous advancement into a body space; a guidewire slidably insertable through a lumen defined by the introducer sheath; an electroporation device configured to be slidably installed through the lumen defined by the introducer sheath, the electroporation device comprising a shaft and a plurality of electrodes disposed at a distal end portion of the shaft, the shaft defining a lumen configured to slidably receive the guidewire; and an occluding vacuum sponge device comprising a shaft defining a lumen to slidably receive the guidewire, the shaft configured to be slidably received within the lumen defined by the shaft of the electroporation device, the occluding vacuum sponge device further comprising an occluding member and a sponge attached to a distal end portion of the shaft.

Such a medical device system may optionally include one or more of the following features. The introducer sheath may be a peel-away sheath introducer. The electroporation device may include a plurality of wire loops extending from the distal end portion of the shaft of the electroporation device. The plurality of electrodes may be attached to the plurality of wire loops. The electroporation device may also include a sheath that is slidably disposed over the shaft of the electroporation device and configured to radially compress the plurality of wire loops to a low-profile configuration within the sheath. The occluding member may be disc-shaped. The sponge may be proximal of the occluding member. The occluding vacuum sponge device may also include a dissolvable sheath disposed over the sponge and the occluding member. The occluding vacuum sponge device may also include one or more drugs integrated within or contained within the sponge.

In another aspect, this disclosure is directed to a method of occluding an anastomosis opening and treating an abscess cavity of a body. The method includes providing and installing any of the medical device systems described herein by: percutaneously installing the introducer sheath so that a distal end portion of the introducer sheath is in the abscess cavity and a proximal end portion of the introducer sheath is exterior of the body; advancing the guidewire through: (i) the lumen defined by the introducer sheath, (ii) the anastomosis opening, (iii) an esophagus, and exiting a mouth of the body; advancing the electroporation device over the guidewire and into the lumen defined by the introducer sheath; deploying the plurality of electrodes in the abscess cavity; and advancing the occluding vacuum sponge device over the guidewire, through the mouth of the body, and through the esophagus until the sponge and occluding member are on opposite sides of the anastomosis opening. The method also includes securing the occluding member against a tissue wall surrounding the anastomosis opening to overlay the anastomosis opening; and delivering electroporation energy via the plurality of electrodes in the abscess cavity.

Such a method may optionally include one or more of the following features. The method may also include delivering irrigation to the abscess cavity via the lumen defined by the shaft of the electroporation device. The method may also include applying suction to the abscess cavity via the lumen defined by the shaft of the electroporation device. The suction may also be applied via the sponge. The method may also include removing the introducer sheath after advancing the electroporation device.

In another aspect, this disclosure is directed to a pleural effusion device that includes: an introducer sheath configured for percutaneous advancement into a body space, the introducer sheath defining a lumen; an electroporation device configured to be slidably installed through the lumen defined by the introducer sheath, the electroporation device comprising a shaft and a plurality of electrodes at a distal end portion of the shaft, the shaft defining a lumen; and a suction tube configured to be slidably advanced within the lumen defined by the shaft of the electroporation device.

Such a pleural effusion device may optionally include one or more of the following features. The electroporation device may also include a plurality of wire loops extending from the distal end portion of the shaft. In some embodiments, the plurality of electrodes comprise, or are attached to, the plurality of wire loops.

In another aspect, this disclosure is directed to a vacuum drain device that includes: a tube comprising a linear portion and a curved portion extending from a distal end of the linear portion, the tube defining a lumen, the curved portion defining a plurality of openings through a wall of the tube and into the lumen; and a plurality of electrodes attached to the curved portion.

Such a vacuum drain device may optionally include one or more of the following features. One or more of the openings may be disposed between adjacent electrodes of the plurality of electrodes. The vacuum drain device may also include a wire mesh surrounding at least a portion of the curved portion. The vacuum drain device may also include a shaft including a distal end portion to which the wire mesh is attached. The shaft may define a lumen configured to slidably receive the tube such that the curved portion can be disposed within an interior space defined by the wire mesh.

Particular embodiments of the subject matter described in this document can be implemented to realize one or more of the following advantages. In some embodiments, the devices described herein are adjustable to accommodate various anatomic shapes and aspects of each body cavity.

Second, in some embodiments the devices and systems can be used to create a broad range of electroporation or pulsed electric field treatments and other therapeutic treatments that are best suited for the individual patient and targeted space.

Third, in some embodiments the devices and systems described herein can deliver a pre-pulse that may serve to numb the nerves, allowing for a painless application of energy to the tissue.

Fourth, in some embodiments the devices and systems described herein can perform thermal and/or non-thermal energy delivery (e.g., RF or pulsed DC electric field ablations) and or delivery light of photobiomodulation therapy to inhibit bacterial, fungi, or viral growth. Through its versatile and adaptive design, the use of the electroporation therapy catheter devices described herein have the potential to significantly enhance the efficacy and ease of performing treatments of abscess cavities and openings in body organs and tissues.

Fifth, in some embodiments electroporation to treat abscess cavities can be delivered in a minimally invasive fashion using the devices and methods provided herein. Such minimally invasive techniques can reduce recovery times, patient discomfort, and treatment costs. In some embodiments, these devices can be delivered endoscopically, percutaneous, surgically, or through a hybrid approach.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a flowchart that illustrates a first example method of treating a GI tract anastomosis in accordance with some embodiments.

FIG. 2 is a perspective view of an example percutaneous introducer sheath in accordance with some embodiments.

FIG. 3 is a perspective view of an example electroporation device in accordance with some embodiments.

FIG. 4 is a perspective view of an example occluding vacuum sponge device in accordance with some embodiments. In some iterations, the sponge is optional, and the flexible catheter provides suction and vacuum through designated holes.

FIG. 5 is a perspective view of the electroporation device of FIG. 3 in a coupled configuration with the occluding vacuum sponge device of FIG. 4.

FIG. 6 is a schematic illustration of the instrument assembly of FIG. 5 in vivo occluding an opening and treating an abscess cavity.

FIG. 7 illustrates another type of occluding vacuum device in accordance with some embodiments. This iteration does not include a sponge.

FIG. 8 is an enlarged perspective view of the distal end portion of the occluding vacuum device of FIG. 7.

FIG. 9 shows the occluding vacuum device of FIG. 7 coupled with the electroporation device of FIG. 3 in vivo occluding an opening and treating an abscess cavity.

FIG. 10 is a perspective view of an example pleural or peritoneal effusion device in accordance with some embodiments.

FIG. 11 is an illustration of an example percutaneous vacuum drainage device in accordance with some embodiments.

FIG. 12 is an enlarged perspective view of the distal end portion of the percutaneous vacuum drainage device of FIG. 11.

FIG. 13 is an illustration of another example percutaneous vacuum drainage device in accordance with some embodiments.

Like reference numbers represent corresponding parts throughout.

### DETAILED DESCRIPTION

This document describes devices and systems for delivering electroporation therapy or pulsed electric field and methods for their use. For example, this document describes devices, systems, and methods for delivering electroporation and adjunct therapies to treat anastomotic leaks and the resulting abscess cavities around the GI tract, within the thoracic cavity, and elsewhere within the human body. In addition, the devices and systems described herein can be used to approximate a hollow structure within the thoracic or abdominal cavities, such as pleural or peritoneal space to apply a pulsed electric field and/or light therapy that can decrease or eliminate the infectious load within the structure and/or enhance the adhesion and collapse of the structure.

In some embodiments, these devices and systems described herein may be also used to treat open wounds. Using an adaptive design, the device may be placed around or over the wound. Such a device will comprise a non-tissue adherent material (e.g., porous, etc.) that will be formable to accommodate various wound sizes and configurations. The sponge may have electrodes, suction tubing (irrigation, etc.), or drugs embedded within, and another form may have electrodes placed on the outside of the sponge. Through its versatile and adaptive design, the catheter devices that use or deliver electroporation, suction, and/or drug delivery therapy, as described herein, have the potential to enhance significantly the efficacy and ease of performing treatments for diabetic ulcers, fistulas, large open wounds, and the like.

The thermal or non-thermal electroporation therapy can be delivered using the devices, systems, and methods described herein. In some embodiments, the electroporation energy is delivered to achieve sterilization and promote healing of spaces such as abscess cavities, diabetic ulcers, fistulas, large open wounds, and the like. In the following description, abscess cavities will be used as an example treatment context. However, it should be understood that the example of abscess cavities is non-limiting and many other treatment contexts (e.g., diabetic ulcers, fistulas, large open wounds, and the like) are also envisioned as treatable by the devices, systems, and methods described herein.

Electroporation can induce transfection of cells using a variety of vectors. It has also been used in oncology for the purpose of cell-specific destruction (e.g., of tumor cells). An advantage of electroporation techniques lies in the potential for cell-specificity. For example, when a voltage is applied to a specific cellular milieu, the phospholipid bilayer of the cell permeabilizes depending on the size of the electric field to which it is exposed. Different cells have different bilayer components, thus resulting in differing electric field thresholds in terms of the size of the electric field required to induce a certain degree of membrane permeabilization. The larger or stronger the electric field, the more likely a cell membrane is to permeabilize to such an extent as to overcome the cell's intrinsic ability to repair the membrane. Accordingly, electroporation can be categorized into two approaches: reversible electroporation (which does not have the goal of cell death, but the goal of cell membrane permeabilization for the purpose of delivery of specific vectors, drugs, etc.) and irreversible electroporation (which has the goal of cell death achieved by sufficient membrane permeabilization as to initiate the apoptosis cascade).

The inventors have discovered abscess and cavity electroporation devices and methods that, even at energy levels required to induce cell death, the energy levels can be controlled so that there is no effect on surrounding structures such as arteries, nerves, other tissues, and the like.

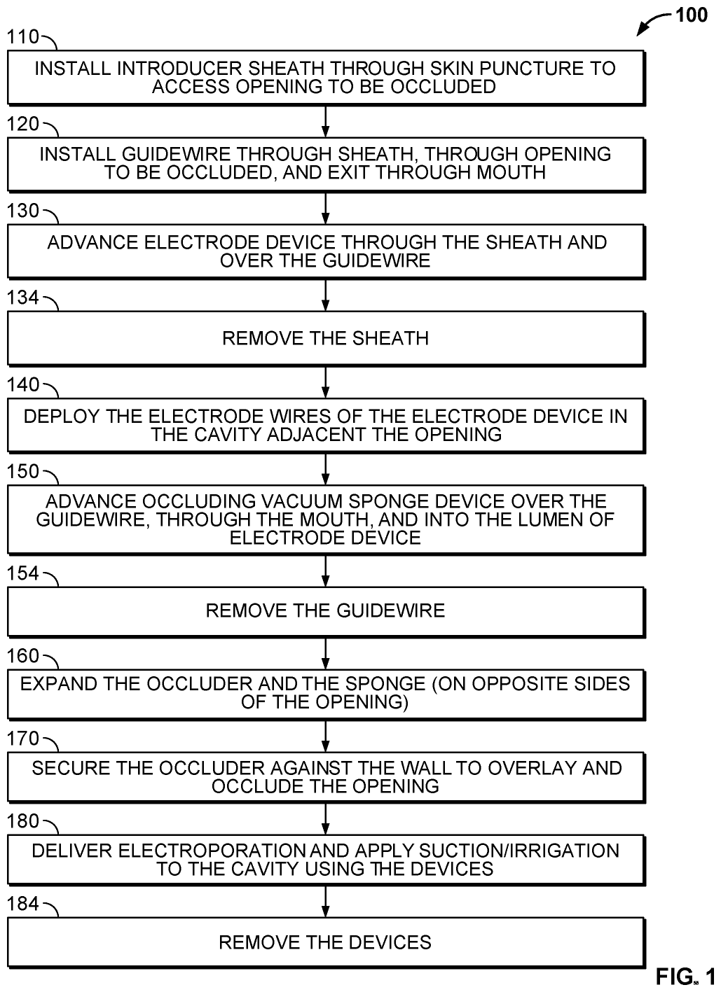

Referring to FIG. 1, an example method 100 describes how a medical device system can be used to treat the body of a patient that has an opening in a GI tract and an abscess cavity adjacent to the opening. The method 100 is described in the context of treating an opening of a stomach of the patient. However, it should be understood that the opening can be in other areas of the GI tract such as, but not limited to, the esophagus, the small intestine, and the colon. Moreover, the method 100 can also be used to treat other types of wounds, ulcers, fistulas, and openings in body organs or tissues (e.g., the lungs, peritoneal space, pleural space, etc.).

While the method 100 does not specifically describe the use of endoscopic ultrasound probes in conjunction with the medical device system, it should be understood that, in some cases, such probes and/or other access and/or visualization systems can be advantageously compatible with the medical device systems described herein.

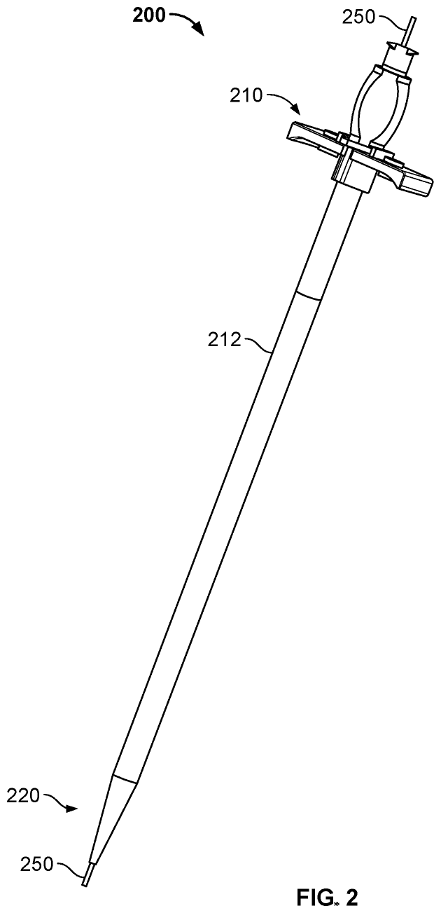

In step 110 of the method 100, an introducer sheath configured for percutaneous advancement is installed through a skin puncture of the patient's body. The introducer sheath is advanced until a distal end portion of the introducer sheath is located in the abscess cavity adjacent to the opening that needs to be closed. FIG. 2 provides an illustration of an example introducer sheath 200 that can be used for this purpose. The introducer sheath 200 includes a proximal end portion 210, a shaft 212, and a distal end portion 220. The proximal end portion 210 remains exterior to the body of the patient. In some embodiments, the introducer sheath 200 is a peel-away sheath or split-able introducer that can be separated apart to facilitate its removal from the body. In some embodiments, the sheath 200 can include a low energy laser, or light emitting diodes (LED) emitting light of different wave lengths, such as blue or violate light at a fluency that is antimicrobial and able to inhibit or treat infection within the collection as an adjunct method. The sheath 200 can be connected to an external generator that modules the wavelength of the light emitted from near infrared to violate, enable pulsed or continuous applications of the light, and titrate the fluence in range between 1 to 500 joules/cm². An example of the emitted light is blue light at a wavelength between 405-470 nm, which as shown anti-microbial activity again gram positive, gram negative, fungi, and/or viruses.

The introducer sheath 200 defines a longitudinal lumen that can slidably receive a guidewire 250. Said another way, a guidewire 250 can be slidably insertable through the lumen defined by the introducer sheath 200.

Still referring to FIG. 1, in step 120 of the method 100 a guidewire (such as the guidewire 250 in FIG. 2) is advanced through the introducer sheath 200. The guidewire is advanced to exit the distal tip of the introducer sheath 200 and to pass through the opening in the GI tract (e.g., the stomach wall). Further, the guidewire 250 is advanced through the patient's esophagus, and exits through the mouth of the patient. Now, both ends of the guidewire 250 are exterior of the body, and the guidewire 250 extends through the body.

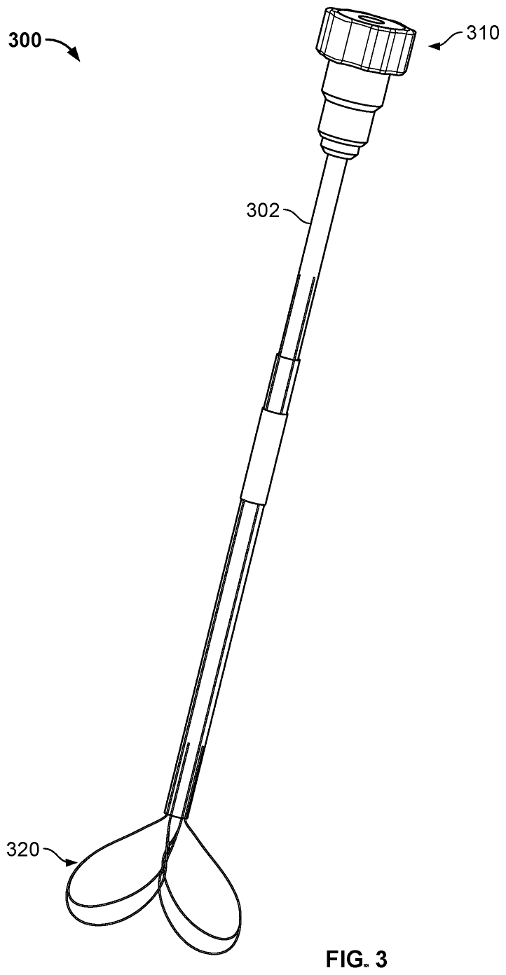

In step 130 of the method 100, an electrode device is advanced into the lumen defined by the introducer sheath 200 and over the guidewire 250. FIG. 3 illustrates an example electrode device 300 that can be used for this purpose. The electrode device 300 includes a proximal end portion 310 that remains exterior of the patient's body, a shaft 302 extending distally from the proximal end portion 310, and a plurality of electrodes 320 disposed at a distal end portion of the shaft 302. The plurality of electrodes 320 can be configured to deliver bipolar, monopolar, or omni-polar electroporation and/or ablation energy. The pulsed electric field can be in the nano, micro, or milli-seconds range.

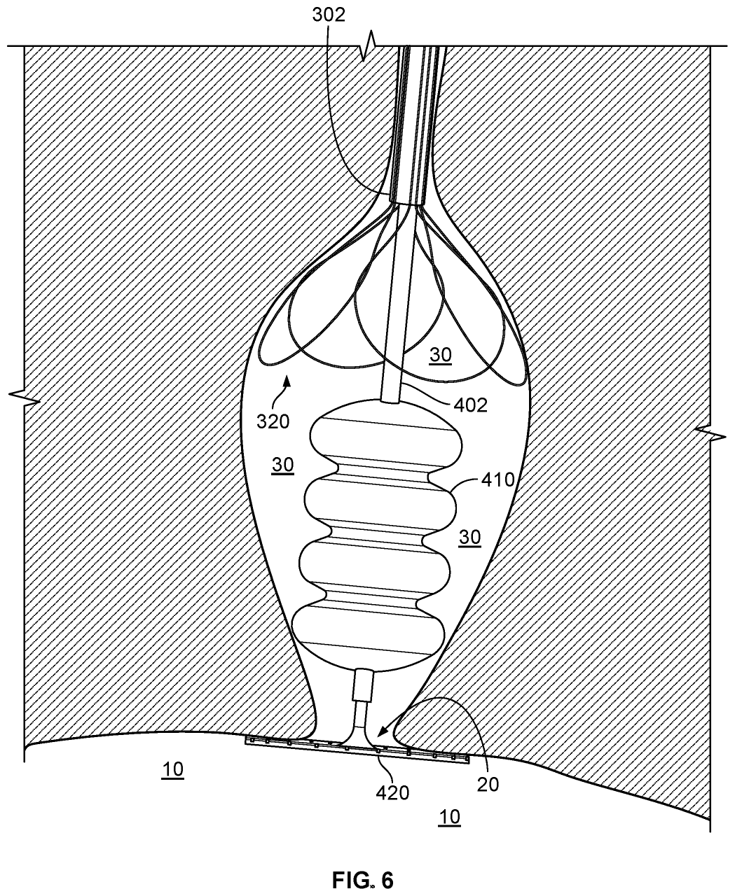

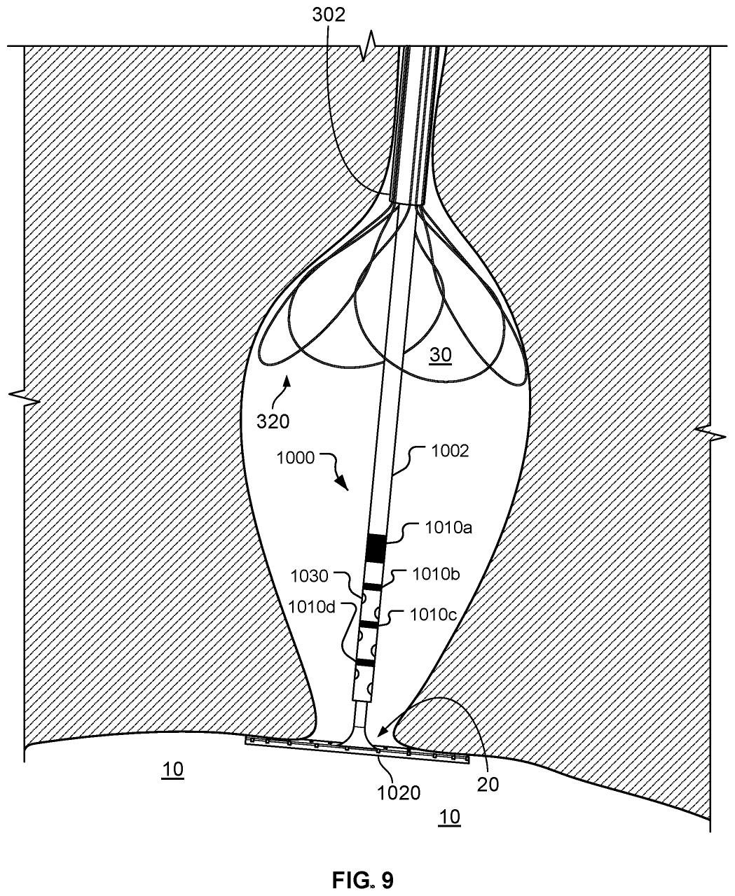

In the depicted embodiment, the plurality of electrodes 320 comprise, or are disposed on, a plurality of wire loops extending from the distal end portion of the shaft 302. The plurality of wire loops can be flexible and reconfigurable between an expanded configuration (as shown in FIG. 3) and a low-profile configuration in which the wire loops are radially compressed by a retractable sheath. In some embodiments, the electrode device 300 includes the sheath that is configured to radially compress the plurality of wire loops to the low-profile configuration within the retractable sheath. The wire loops can be deployed to the expanded configuration while in the abscess cavity adjacent to the GI opening (e.g., see FIGS. 6 and 9).

Still referring to FIG. 1, in step 134 of the method 100, the introducer sheath 200 can be removed from the body (while the guidewire 250 and the electrode device 300 remain in their respective positions and orientations within the body. In some embodiments, the introducer sheath 200 can be removed from the body by being longitudinally separated into two or more pieces (so that the introducer sheath 200 can be removed while the guidewire 250 and the electrode device 300 remain). In some embodiments, the introducer sheath 200 can be a peel-away sheath introducer to make it suitable for this purpose and functionality.

In step 140 of the method 100, if not already completed, the wire loops of the electrode device 300 can be deployed to the expanded configuration while in the abscess cavity adjacent to the GI opening. To perform this step, in some embodiments a retractable sheath of the electrode device 300 can be retracted proximally to deploy the wire loops.

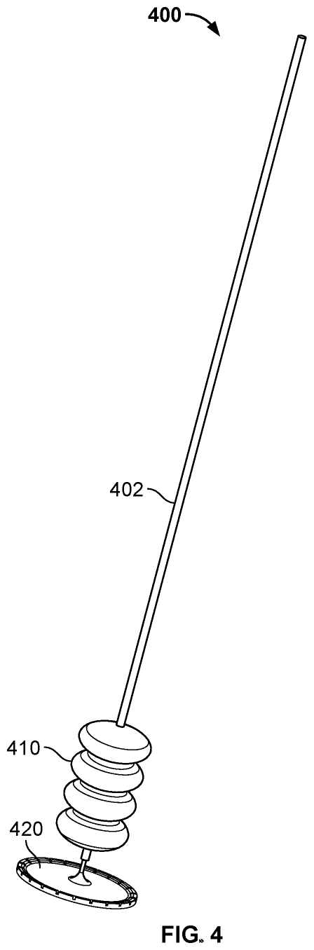

In step 150 of the method 100, an occluding vacuum sponge device 400, such as depicted in FIG. 4 (but with a dissolvable sheath that is not illustrated), can be advanced over the guidewire 250 through the mouth and esophagus of the patient, and into the stomach. The dissolvable sheath radially constrains the sponge to allow a low-profile delivery. The dissolvable sheath can be made of material such as cellulose that dissolves upon contact with water.

The example occluding vacuum sponge device 400 comprises a flexible shaft 402 defining a lumen to slidably receive the guidewire 250. The occluding vacuum sponge device 400 further comprises an occluding member 420 and a sponge 410 attached to a distal end portion of the shaft 402. The occluding member 420 and the sponge 410 are radially compressible between a deployed configuration (as shown) and a low profile configuration when radially restrained within a sheath. In some embodiments, the sheath is dissolvable or biodegradable. Accordingly, the sheath used on the occluding vacuum sponge device 400 to radially constrain the occluding member 420 and the sponge 410 during advancement is configured to break apart in situ so that the occluding member 420 and the sponge 410 will be allowed to radially expand in situ. In some embodiments, this dissolvable sheath may contain drugs integrated within or contained within the sponge 410.

The occluding member 420 can be constructed in various ways. In some embodiments, the occluding member 420 is a made of a super-elastic Nitinol frame with an attached covering/occluding material. In particular embodiments, the occluding member 420 is a made of one or more elastic materials such as, but not limited to, latex, silicon, and the like. In the depicted embodiment, the occluding member 420 is disc-shaped. Alternatively, the occluding member 420 can have other shapes such as, but not limited to, spherical, cylindrical, conical, frustoconical, oblong, and so forth.

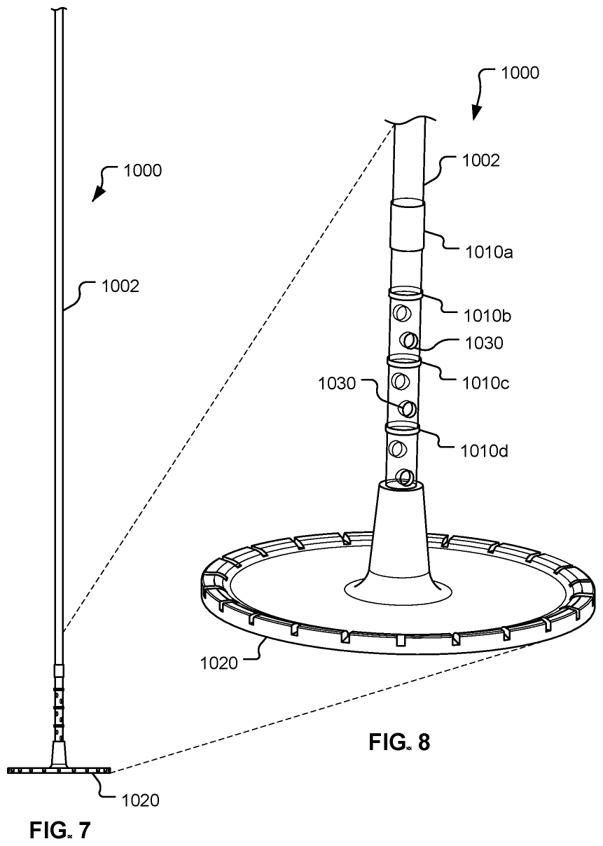

The sponge 410 can have various configurations. In the depicted embodiment, the sponge 410 includes multiple bulbous portions that are adjacent to each other. In some embodiments, the sponge 410 is configured as a single cylindrical shape (or as a helical shape). The sponge 410 is optional (e.g., see FIGS. 7-9). In some embodiments, the flexible catheter provides suction and vacuum through designated holes.

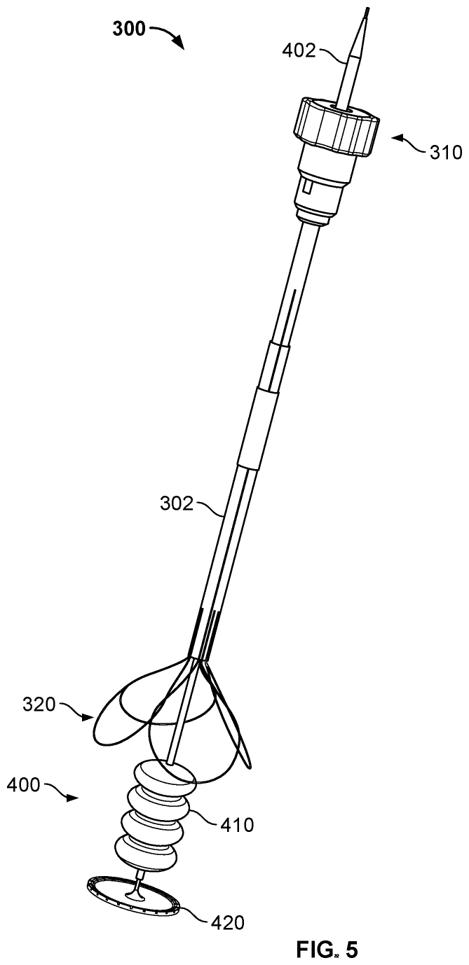

The end of the shaft 402 that is opposite of the occluding member 420 and the sponge 410 is advanced through the mouth first. As the advancement is continued, the shaft 402 continues following the guidewire 250 right into the lumen defined by the shaft 302 of the electroporation device 300. When that has taken place, the configuration of the electroporation device 300 and the occluding vacuum sponge device 400 is as shown in FIG. 5.

Still referring to FIG. 1, in step 154 of the method 100, the guidewire 250 is removed from the patient (and from within the electroporation device 300 and the occluding vacuum sponge device 400).

In step 160 of the method 100, the occluding member 420 and the sponge 410 expand on opposite sides of the GI tract opening. This is arrangement illustrated in FIG. 6 in which a stomach space 10, a stomach wall opening 20, and an abscess cavity 30 are depicted. The occluding member 420 overlays the opening 20. The sponge 410 and the plurality of electrodes 320 are in the abscess cavity 30. In some embodiments, to allow the occluding member 420 to expand, an elastic band-like restraining member can be cut in situ. In some embodiments, to allow the sponge 410 to expand, the aforementioned dissolvable sleeve (which can be made of a material such as cellulose in some embodiments) can be allowed to dissolve. An irrigate solution can be added to accelerate the dissolving in some cases.

In step 170 of the method 100, the occluding member 420 is drawn securely snug against the inner wall surface of the stomach space 10. In that arrangement, the proximal end portion 310 of the electrode device 300 can be secured to the shaft 402 of the occluding vacuum sponge device 400. The proximal end portion 310 of the electrode device 300 can include a lockable knob for this purpose.

In step 180 of the method 100, electroporation can be periodically delivered (e.g., one or more times per day) to the abscess cavity 30 via the plurality of electrodes 320. The electroporation energy from plurality of electrodes 320 can sterilize the abscess cavity 30 to promote healing of the abscess cavity 30. The electroporation energy (e.g., one or more pulse trains of nanosecond pulses ranging from 50 nanoseconds pulses up to 100 microseconds pulses, and having a voltage of 500 volts DC to 15 kilovolts DC) can eliminate sepsis in the abscess cavity. Gradually, over time (e.g., approximately 1-3 weeks), the abscess cavity 30 will heal and close.

In addition, step 180 includes the application of suction and irrigation to the abscess cavity 30. The suction and irrigation can be applied via the lumen of the shaft 302 of the electroporation device 300 and/or via one or more openings in the shaft 402 of the occluding vacuum sponge device 400. In some embodiments, suction and/or irrigation is applied via the sponge 410. Such suction and/or irrigation promotes the healing of the abscess cavity 30, and/or the healing (e.g., closure) of the opening 20, in some cases.

In step 184 of the method 100, the devices are removed from the body. In some embodiments, the occluding member 420 is separated from the shaft 402 and remains attached (e.g., via fibrosis) to the wall of the stomach. In some embodiments, the occluding member 420 is separated from the shaft 402 and is naturally expelled from the body.

FIGS. 7 and 8 illustrate another type of occluding device 1000. In some embodiments, the occluding device 1000 is configured similarly to, and can be used similarly to, the occluding vacuum sponge device 400 (but without the sponge 410).

The occluding device 1000 includes a flexible shaft 1002 defining a central longitudinal lumen configured to slidably receive the guidewire 250. In the depicted embodiment, the wall of the distal end portion of the flexible shaft 1002 defines one or more fenestrations 1030. The fenestrations 1030 serve as ports to the lumen of the flexible shaft 1002. Accordingly, the fenestrations 1030 can be used to deliver suction and/or irrigation to the area surrounding the distal end portion of the flexible shaft 1002 (e.g., as described above in reference to step 180).

The occluding device 1000 also includes one or more electrodes attached to the distal end portion of the flexible shaft 1002. In the depicted embodiment, there are four electrodes 1010a, 1010b, 1010c, and 1010d attached to the flexible shaft 1002 in a spaced-apart manner. In the depicted embodiment, at least some of the fenestrations 1030 are located between some of the electrodes 1010a-d.

While the depicted embodiment includes the four electrodes 1010a-d, in some embodiments other numbers of electrodes can be included on the occluding device 1000, such as one, two, three, five, six, seven, eight, nine, ten, and more than ten.

The electrodes can be, individually, any desired size. For example, in the depicted embodiment the electrode 1010a is illustrated as being larger than the other electrodes 1010b-d. However, any combination of sizes of the electrodes can be included on the distal end portion of the flexible shaft 1002. In some embodiments, all of the electrodes are the same size as each other.

In some embodiments, these electrodes 1010a-d are operable independently of each other. That is, each of the electrodes 1010a-d can be selectively operated as an anode or a cathode (or both in a reversible manner), in any arrangement that the clinician user desires (depending on what is best for the particular usage context). In some embodiments, the electrodes 1010a-d can be operated in pairs (with one electrode being an anode and the other electrode being a cathode) and any other desired configuration, arrangement, iteration, or permutation. For example, in some embodiments the electrode 1010a can be operated as an anode and each of the electrodes 1010b-d can be operated as a cathode (either simultaneously or sequentially).

The occluding device 1000 also includes an occluding member 1020 attached to a distal end portion of the shaft 1002. The occluding member 1020 is radially compressible between a deployed configuration (as shown) and a low profile configuration when radially restrained within a sheath. In some embodiments, the sheath is dissolvable or biodegradable. Accordingly, the sheath used on the occluding device 1000 to radially constrain the occluding member 1020 during advancement is configured to break apart in situ so that the occluding member 1020 will be allowed to radially expand in situ. In some embodiments, a manually removable radial restraining device is used to radially constrain the occluding member 1020 during advancement. Then, the clinician user can remove the radial restraining device to allow the occluding member 1020 to radially self-expand in situ.

The occluding member 1020 can have any of the shapes and can be constructed in any of the configurations as described above in reference to the occluding member 420.

To deploy the occluding device 1000, in some embodiments the end of the shaft 1002 that is opposite of the occluding member 1020 is advanced through the mouth first (e.g., as described above regarding step 150). As the advancement is continued, the shaft 1002 continues following the guidewire 250 right into the lumen defined by the shaft 302 of the electroporation device 300. When that has taken place, the configuration of the electroporation device 300 and the occluding device 1000 can be, for example, as shown in FIG. 9.

In some embodiments, the treatment is provided using the occluding device 1000 without using, or even deploying, the electroporation device 300. However, in some embodiments the electrodes 320 of the electroporation device 300 can be used in cooperation with the electrodes 1010a-d of the occluding device 1000. That is, in some embodiments the electrodes 320 can be operated as cathodes and the electrodes 1010a-d can be operated as anodes (or the opposite arrangement). Alternatively, in some embodiments the electrodes 320 can be a combination of anodes and cathodes and the electrodes 1010a-d can be a combination of anodes and cathodes. The electrodes 320 can be energized simultaneously or sequentially with the electrodes 1010a-d.

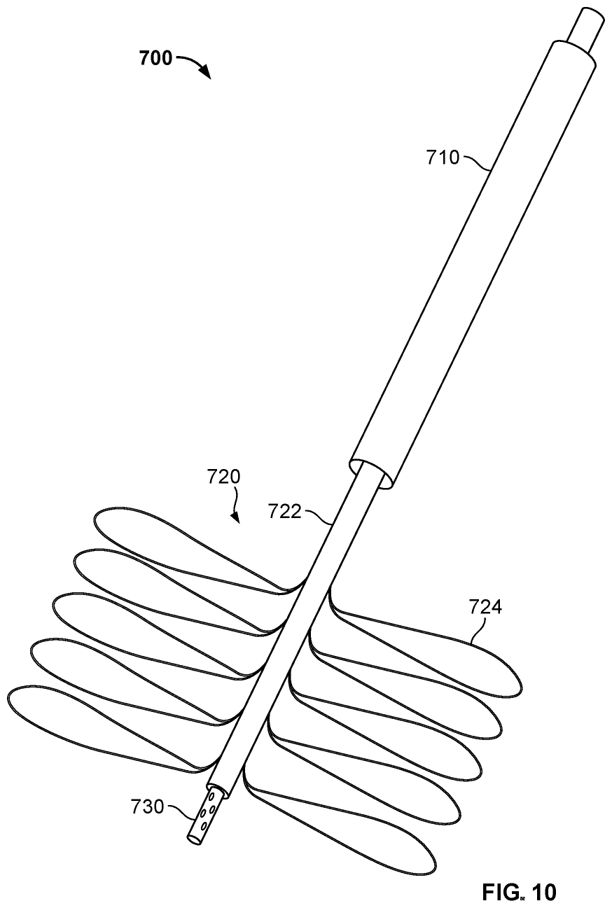

FIG. 10 depicts another type of medical device system. In particular, an example pleural effusion device system 700 is depicted. The pleural effusion device system 700 comprises: (i) an introducer sheath 710 configured for percutaneous advancement into a body space, the introducer sheath 710 defining a lumen; (ii) an electroporation device 720 configured to be slidably installed through the lumen defined by the introducer sheath 710, the electroporation device 720 comprising a shaft 722 and a plurality of electrodes 724 (e.g., one or more electrodes disposed on a plurality of wire loops) at a distal end portion of the shaft 722, the shaft 722 defining a lumen; and (iii) a suction tube 730 configured to be slidably advanced within the lumen defined by the shaft 722 of the electroporation device 720.

The pleural effusion device system 700 can be used to deliver suction and electroporation to an open pleural space that needs healing, for example. In some such cases, the electroporation delivered by the electroporation device 720 can cause inflammation of the cells of the pleural layer and the visceral layer. Suction can also be delivered to the pleural space. Then, as the inflamed cells of the pleural layer and the visceral layer heal (while being drawn into contact with each other via the suction from the suction tube 730, the cells of the pleural layer and the visceral layer can join together to close the pleural space. In some embodiments, a low energy laser, or light emitting diodes (LED) probe can be introduced through the central lumen of the device or mounted on the shaft of the device, on mounted on the inner wall of the lumen of the device. As such, light of different wavelengths, such as blue or violate light at a fluency that is antimicrobial and able to inhibit or treat infection within the collection as an adjunct method. The probed can be connected to an external generator that modules the wavelength of the light emitted from near infrared to violate, enable pulsed or continuous applications of the light, and titrate the fluence in range between 1 to 500 joules/cm². An example of the emitted light is blue light at a wavelength between 405-470 nm, which as shown anti-microbial activity again gram positive, gram negative, fungi, and viruses.

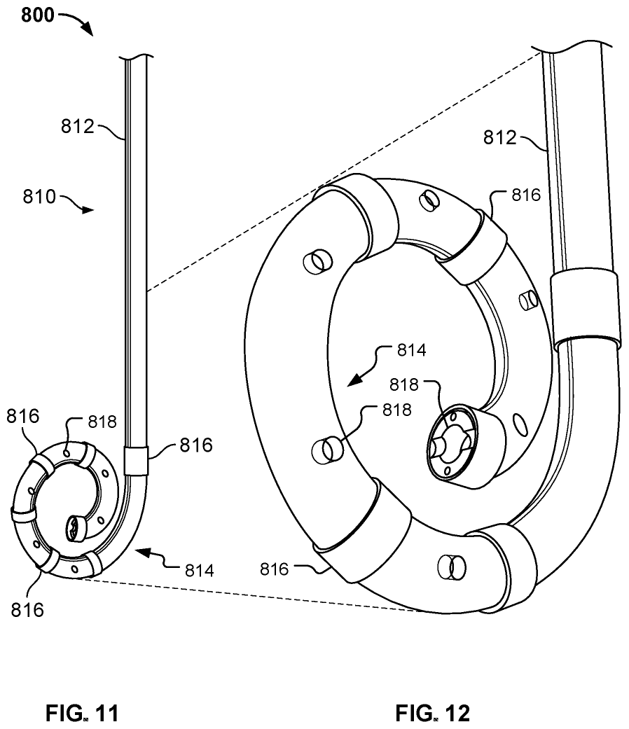

FIGS. 11 and 12 depict an example vacuum drain device 800. FIG. 12 is an enlarged view of the distal end portion of the vacuum drain device 800.

In the depicted embodiment, the vacuum drain device 800 is configured to deliver suction/irrigation and electroporation. The vacuum drain device 800 is used in any body cavity or location that needs drainage and healing. The vacuum drain device 800 can be installed percutaneously or through a natural body orifice. In some embodiments, the vacuum drain device 800 is installed over a guidewire, through a working channel of a scope, or through an introducer sheath or stiffening device. In some embodiments, two or more of the vacuum drain devices 800 can be installed to the same body cavity space.

The vacuum drain device 800 includes a tube 810 comprising a linear portion 812 and a curved portion 814 extending from a distal end of the linear portion 812. The tube 810 defines a lumen through which suction and/or irrigation can be administered in vivo via one or more fenestrations 818. The curved portion 814 defines a plurality of fenestrations 818 through a wall of the tube 810 and into the lumen. The plurality of fenestrations 818 are used to administer the suction and/or irrigation.

The vacuum drain device 800 also includes a plurality of electrodes 816 attached to the curved portion 814. The electrodes 816 can be energized to deliver electroporation and/or ablation energy within the body space. In some embodiments, each of the electrodes 816 is independently wired and independently controllable.

The suction, fluid drainage, and electroporation delivered from the vacuum drain device 800 can promote healing and/or closure of body cavities with wounds (e.g., from surgery or otherwise), such as in and around the lungs and other thoracic and/or abdominal spaces. In some embodiments, the drain can include a low energy laser, or light emitting diodes (LED) emitting light of different wave lengths, such as blue or violate light at a fluency that is antimicrobial and able to inhibit or treat infection within the collection as an adjunct method. The drain can be connected to an external generator that modules the wavelength of the light emitted from near infrared to violate, enable pulsed or continuous applications of the light, and titrate the fluence in range between 1 to 500 joules/cm². An example of the emitted light is blue light at a wavelength between 405-470 nm, which as shown anti-microbial activity again gram positive, gram negative, fungi, and viruses. In some embodiments, the vacuum drain device 800 is configured to deliver the LED light and is not equipped with the electrodes 816. In some embodiments, the vacuum drain device 800 is configured to deliver the LED light and is equipped with the electrodes 816.

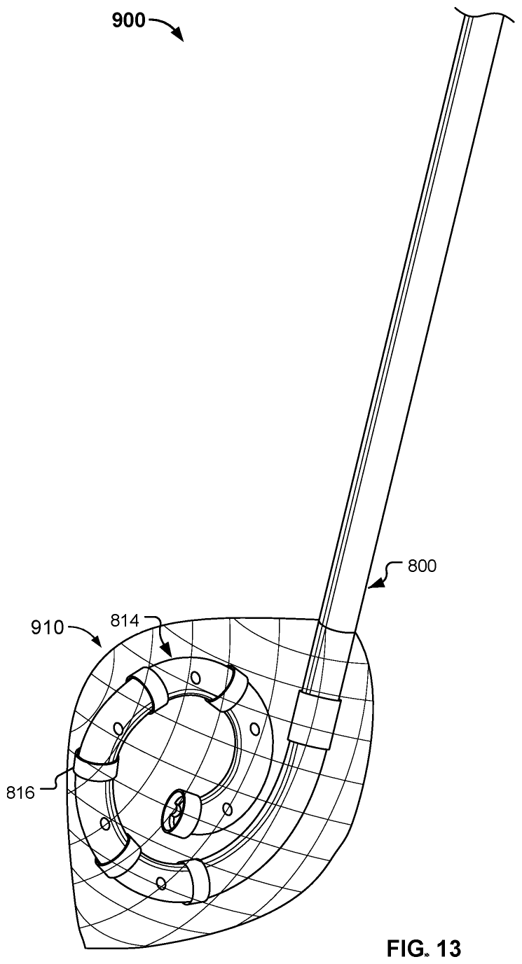

FIG. 13 depicts another example vacuum drain device 900. The vacuum drain device 900 is configured to deliver suction and electroporation. The vacuum drain device 900 is used in any body cavity or location that needs drainage and healing. The vacuum drain device 900 can be installed percutaneously or through a natural body orifice. In some embodiments, the vacuum drain device 900 is installed over a guidewire, through a working channel of a scope, or through an introducer sheath. In some embodiments, two or more of the vacuum drain devices 900 can be installed to the same body cavity space.

The vacuum drain device 900 is the same as the vacuum drain device 800 with the addition of a second shaft including a distal end portion to which a wire mesh 910 is attached. The shaft 810 of the vacuum drain device 800 can be slidably disposed within a lumen defined by the second shaft to which the wire mesh 910 is attached.

When deployed (as shown), the wire mesh 910 surrounds at least a portion of the curved portion 814. The wire mesh 910 can be made of Nitinol or stainless steel in some embodiments. Accordingly, the wire mesh 910 can act as an electrode for the electroporation delivery (e.g., as an anode or a cathode) in conjunction with the electrodes 816.

In some embodiments, the wire mesh 910 is manually expandable (e.g., to the depicted configuration) and retractable to a low-profile configuration.

### Additional Optional Embodiments, Features and Uses

While in the depicted embodiments the electrodes are DC electrodes, alternatively, or additionally, some embodiments of electroporation catheters can be configured to deliver other types of electroporation energy such as, but not limited to, radiofrequency (RF), AC, cryogenic, chemical, and the like. In some embodiments, a combination of such energy sources can be used within a single embodiment of intravascular electroporation catheter (e.g., RF and DC are used in combination is some embodiments). The electroporation energy can be omnipolar, monopolar or bipolar. In some implementations, two or more types of electroporation energy sources can be coupled to electrodes. In some embodiments, a low energy laser, or light emitting diodes (LED) emitting light of different wavelengths are introduced or connected to these devices. These light sources can deliver light, such as blue or violate light, at a fluency that is antimicrobial and able to inhibit or treat infection within the collection as an adjunct method. The light source can be an external generator that modules the wavelength of the light emitted from near infrared to violate, enable pulsed or continuous applications of the light, and titrate the fluence in range between 1 to 500 joules/cm². An example of the emitted light is blue light at a wavelength between 405-470 nm, which as shown anti-microbial activity again gram positive, gram negative, fungi, and viruses.

In some embodiments, the devices described herein are adjustable to accommodate various anatomic shapes and aspects of each body space cavity.

In some embodiments, the devices and systems can be used to create a broad range of ablation lesion sets that are best suited for the individual patient.

In some embodiments, the devices and systems described herein can deliver a pre-pulse that may serve to numb the nerves, allowing for a painless application of energy to the tissue.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A medical device system comprising:
an introducer sheath configured for percutaneous advancement into a body space;
a guidewire slidably insertable through a lumen defined by the introducer sheath; and
an occluding vacuum device comprising a shaft and a occluding member attached to a distal end of the shaft, the shaft defining a lumen to slidably receive the guidewire and defining one or more fenestrations through a wall of the shaft, the occluding vacuum device further comprising one or more electrodes attached to the shaft.

2. The medical device system of claim 1, wherein at least some of the one or more fenestrations are located between two electrodes of the one or more electrodes.

3. The medical device system of claim 1, wherein the occluding member is disc-shaped.

4. The medical device system of claim 3, wherein the occluding member is reconfigurable between a low-profile delivery configuration and a radially expanded deployed configuration.

5. A medical device system comprising:
an introducer sheath configured for percutaneous advancement into a body space;
a guidewire slidably insertable through a lumen defined by the introducer sheath;
an electroporation device configured to be slidably installed through the lumen defined by the introducer sheath, the electroporation device comprising a shaft and a plurality of electrodes disposed at a distal end portion of the shaft, the shaft defining a lumen configured to slidably receive the guidewire; and
an occluding vacuum sponge device comprising a shaft defining a lumen to slidably receive the guidewire, the shaft configured to be slidably received within the lumen defined by the shaft of the electroporation device, the occluding vacuum sponge device further comprising an occluding member and a sponge attached to a distal end portion of the shaft.

6. The medical device system of claim 5, wherein the introducer sheath is a peel-away sheath introducer.

7. The medical device system of claim 5, wherein the electroporation device comprises a plurality of wire loops extending from the distal end portion of the shaft of the electroporation device.

8. The medical device system of claim 7, wherein the plurality of electrodes are attached to the plurality of wire loops.

9. The medical device system of claim 7, wherein the electroporation device further comprises a sheath that is slidably disposed over the shaft of the electroporation device and configured to radially compress the plurality of wire loops to a low-profile configuration within the sheath.

10. The medical device system of claim 5, wherein the occluding member is disc-shaped.

11. The medical device system of claim 10, wherein the sponge is proximal of the occluding member.

12. The medical device system of claim 11, wherein the occluding vacuum sponge device further comprises a dissolvable sheath disposed over the sponge and the occluding member, and further comprising one or more drugs integrated within or contained within the sponge.

13. A method of occluding an anastomosis opening and treating an abscess cavity of a body, the method comprising:
providing and installing the medical device system of claim 1 by:
percutaneously installing the introducer sheath so that a distal end portion of the introducer sheath is in the abscess cavity and a proximal end portion of the introducer sheath is exterior of the body;
advancing the guidewire through: (i) the lumen defined by the introducer sheath, (ii) the anastomosis opening, (iii) an esophagus, and exiting a mouth of the body;
advancing the electroporation device over the guidewire and into the lumen defined by the introducer sheath;
deploying the plurality of electrodes in the abscess cavity; and
advancing the occluding vacuum sponge device over the guidewire, through the mouth of the body, and through the esophagus until the sponge and occluding member are on opposite sides of the anastomosis opening;
securing the occluding member against a tissue wall surrounding the anastomosis opening to overlay the anastomosis opening; and
delivering electroporation energy via the plurality of electrodes in the abscess cavity.

14. The method of claim 13, further comprising delivering irrigation to the abscess cavity via the lumen defined by the shaft of the electroporation device.

15. The method of claim 14, further comprising applying suction to the abscess cavity via the lumen defined by the shaft of the electroporation device.

16. The method of claim 15, wherein the suction is also applied via the sponge.

17. The method of claim 13, further comprising removing the introducer sheath after advancing the electroporation device.

18. A pleural effusion device system comprising:
an introducer sheath configured for percutaneous advancement into a body space, the introducer sheath defining a lumen;
an electroporation device configured to be slidably installed through the lumen defined by the introducer sheath, the electroporation device comprising a shaft and a plurality of electrodes at a distal end portion of the shaft, the shaft defining a lumen; and
a suction tube configured to be slidably advanced within the lumen defined by the shaft of the electroporation device.

19. The pleural effusion device system of claim 18, wherein the electroporation device further comprises a plurality of wire loops extending from the distal end portion of the shaft, wherein the plurality of electrodes comprise or are attached to the plurality of wire loops.

20. A vacuum drain device comprising:
a tube comprising a linear portion and a curved portion extending from a distal end of the linear portion, the tube defining a lumen, the curved portion defining a plurality of openings through a wall of the tube and into the lumen; and
a plurality of electrodes attached to the curved portion.

21. The vacuum drain device of claim 20, wherein one or more of the openings are disposed between adjacent electrodes of the plurality of electrodes.

22. The vacuum drain device of claim 20, further comprising a wire mesh surrounding at least a portion of the curved portion.

23. The vacuum drain device of claim 22, further comprising a shaft including a distal end portion to which the wire mesh is attached.

24. The vacuum drain device of claim 23, wherein the shaft defines a lumen configured to slidably receive the tube such that the curved portion can be disposed within an interior space defined by the wire mesh.
